(19) United States
(12) Patent Application Publication
(10) Pub. No.: US 2010/0324625 A1
(43) Pub. Date: Dec. 23, 2010

(54) BRACKETING SCAR FOR TREATING PAIN WITH ELECTRICAL STIMULATION

(75) Inventors: Carl Wahlstrand, North Oaks, MN (US); Dale Seeley, Spring Park, MN (US); Gabi Molnar, Fridley, MN (US); Lisa Johanek, White Bear Lake, MN (US)

Correspondence Address: CAMPBELL NELSON WHIPPS, LLC, 408 ST. PETER STREET, SUITE 240, ST. PAUL, MN, 55102, US

(73) Assignee: MEDTRONIC, INC., Minneapolis, MN (US)

(21) Appl. No.: 12/819,265

(22) Filed: Jun. 21, 2010

Related U.S. Application Data
(60) Provisional application No. 61/218,540, filed on Jun. 19, 2009.

(51) Int. Cl. A61N 1/34

(52) U.S. Cl. 607/46

(57) ABSTRACT

A method includes implanting one or more leads in proximity to a scar of a patient. The one or more leads have a plurality of electrodes and are implanted such that a first electrode of the plurality of electrodes is implanted subcutaneously on a side of a longitudinal axis of the scar and a second electrode of the plurality of electrodes is implanted on an opposing side of the longitudinal axis of the scar. Such an electrode configuration may be achieved by positioning leads transverse or perpendicular to the scar. The method further includes applying electrical signals to tissue in the region of the scar via the first and second electrodes. The electrical signals applied in such a manner may be helpful for treating pain associated with the scar.

### RELATED APPLICATION

This application claims the benefit of priority to U.S. provisional patent application no. 61/218,540, filed Jun. 19, 2009, which provisional application is incorporated herein by reference to the extent that it does not conflict with the present disclosure.

### FIELD

This disclosure relates to implantable medical devices for treating pain.

### BACKGROUND

Implantable electrical signal generators have been used successfully for treating a variety of diseases, including chronic pain. For example, chronic low back an leg pain have been successfully treated using implantable neurostimulator systems that apply electrical signals to selected regions of the spinal cord. While spinal cord stimulation can theoretically be used to treat many types of pain by altering afferent pain signals running through the spinal cord, some types of pain may be difficult to treat via spinal cord stimulation or may result in unintended side effects. In such situations, it may be desirable to apply pain treating electrical signals at peripheral areas closer in proximity to the pain.

Such peripheral nerve stimulation or peripheral nerve field stimulation, as it is often called, does not have as long of a history of use for treatment of pain with implantable medical devices, relative to spinal cord stimulation. Accordingly, methods, techniques and devices for application of electrical signals to the periphery for treating pain are still being developed. For example, new methods, techniques and devices may be needed to ensure that leads for applying electrical signals to peripheral nerves are properly positioned when implanted so that electrical signals emitted from the lead “capture” the desired peripheral nerves.

### BRIEF SUMMARY

The present disclosure describes, among other things, bracketing a scar, such as a surgical scar that can be associated with chronic pain, or other regions of pain with electrodes of leads for applying pain treating electrical signals to the region. The electrodes are implanted along an arcuate path to bracket the scar or other region of pain. For example, the electrodes may be positioned in a wishbone, horseshoe, circular, semicircular, “U” or similar manner. When a nerve trunk associated with the pain to be treated is superficial, as is often the case with pain associated with herniorrhaphy, the electrodes may also bracket the nerve trunk. In any case, leads positioned so that electrodes bracket a region of pain may produce sufficient paresthesia for pain relief

In various embodiments, a method includes implanting one or more leads in proximity to a scar of a patient. The one or more leads have a plurality of electrodes and are implanted such that a first electrode of the plurality of electrodes is implanted subcutaneously on a side of a longitudinal axis of the scar and a second electrode of the plurality of electrodes is implanted on an opposing side of the longitudinal axis of the scar. It will be understood that one or more leads may be positioned transverse to the scar to achieve such an electrode configuration. The method further includes applying electrical signals to tissue in the region of the scar via the first and second electrodes. The electrical signals applied in such a manner may be helpful for treating pain associated with the scar.

In some embodiments, a method for treating pain associated with a scar includes implanting one or more leads in proximity to the scar of a patient. The one or more leads have a plurality of electrodes and are implanted such that a first electrode of the plurality of electrodes is implanted subcutaneously on a side of a longitudinal axis of the scar and a second electrode of the plurality of electrodes is implanted on an opposing side of the longitudinal axis of the scar. Again, one or more leads may be implanted transverse to the scar to accomplish such a configuration of electrodes. The method further includes applying electrical signals to tissue in the region of the scar via the first and second electrodes to treat the pain.

In numerous embodiments, a method for treating post-herniorrhaphy pain includes implanting one or more leads in proximity to a post-surgical herniorrhaphy scar of a patient. The one or more leads have a plurality of electrodes and are implanted such that a first electrode of the plurality of electrodes is implanted subcutaneously on a side of a longitudinal axis of the scar and a second electrode of the plurality of electrodes is implanted on an opposing side of the longitudinal axis of the scar. In some embodiments, one or more leads are implanted transverse to the scar to achieve such an electrode configuration. The method further includes applying electrical signals to tissue in the region of the scar via the first and second electrodes to treat the pain.

These and various other features and advantages will be apparent from a reading of the following detailed description.

### BRIEF DESCRIPTION OF THE DRAWINGS

FIG. 1 is a schematic side view of a generic implantable electrical system.

FIG. 2 is a schematic drawing of showing a generic scar of a patient.

FIGS. 3-5 are a schematic drawing of leads, or distal portions thereof, implanted in subcutaneous tissue and bracketing a scar.

FIGS. 6A-D are schematic drawings illustrating an embodiment of a method for implanting leads to bracket a scar in a manner similar to that shown in FIG. 3.

FIGS. 7A-C are schematic drawings illustrating an embodiment of a method for implanting a bifurcated lead to bracket a scar in a manner similar to that shown in FIG. 4.

FIG. 8A is a schematic perspective view of an embodiment of an arcuate introducer for implanting a lead along an arcuate path in a patient.

FIGS. 8B-D are schematic drawings illustrating an embodiment of a method for implanting a lead to bracket a scar in a manner similar to that shown in FIG. 5 using the introducer depicted in FIG. 8A.

FIGS. 9-12 are schematic drawings of various embodiments of implantable medical systems having leads, or distal portions thereof, implanted in subcutaneous tissue and bracketing a scar.

FIG. 13 is a schematic drawing of leads, or distal portions thereof, implanted in subcutaneous tissue and bracketing a scar and in electrical communication with at least one nerve trunk.

FIG. 14 is a schematic drawing of leads, or distal portions thereof, implanted in subcutaneous tissue and bracketing a scar and in electrical communication with at least one nerve trunk.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the apparatuses, systems and methods described herein. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise.

As used herein, “representative,” “exemplary,” and the like are used in the context of “providing an example” and do not necessarily indicate that the example provided is superior to, or more particularly suited for the intended purpose than, other potential examples.

The present disclosure describes, among other things, implanting electrodes of one or more leads along an arcuate path to bracketing a scar, such as a surgical scar that can be associated with chronic pain, or other region of interest so that pain treating electrical signals may be effectively applied to the scar region or other region of interest. The electrodes may be positioned in a wishbone, horseshoe, semicircular, circular, semi-oval, oval, semi-elliptical, elliptical, helical, “U” or similar manner bracketing or circling at least a portion of the scar to apply the electrical therapy. When nerve trunks associated with the region of pain are superficial, such as often occurs with pain associated with herniorrhaphy or thoracotomy, the electrodes may also bracket the nerve trunks. In any case, electrical signals applied via electrodes positioned to bracket a scar or other region of pain may produce sufficient paresthesia for pain relief

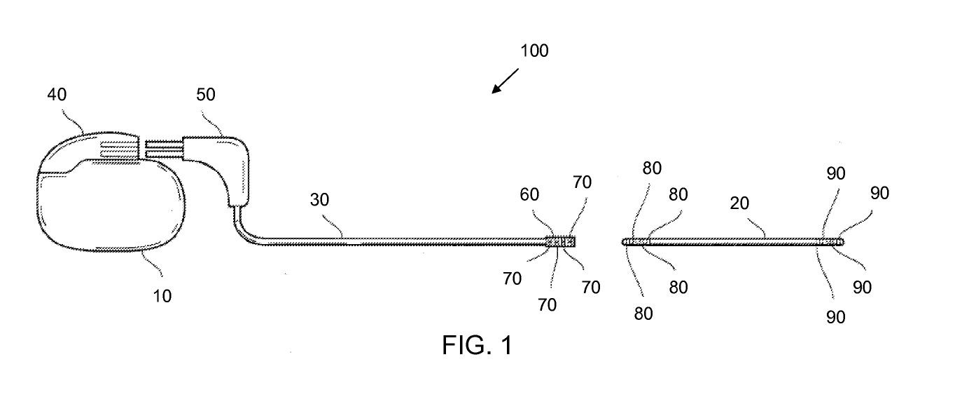

Any suitable electrical signal generator system may be employed for applying electrical signals in such a manner. For example and with reference to FIG. 1, a representative implantable system 100 is having an electrical signal generator 10 is shown. The system 100 also includes a lead extension 30 and a lead 20. Implantable electrical signal generator 10 includes a connector header 40 configured to receive plug 50 at proximal end of lead extension 30 or other adaptor to couple lead 20 to electrical signal generator 10. The distal end portion of lead extension 30 includes a connector 60 configured to receive proximal end portion of lead 20. Connector 60 includes internal electrical contacts 70 configured to electrically couple extension 30 to lead 20 via electrical contacts 80 disposed on the proximal end portion of lead 20. Electrodes 90 are disposed on distal end portion of lead 20 and are electrically coupled to electrical contacts 80, typically through conductors (not shown). In general, a lead 20 may include any number of electrodes 90, e.g. one, two, three, four, five, six, seven, eight, or sixteen. Typically, each electrode 90 is electrically coupled to a discrete electrical contact 80. While not shown, it will be understood that more than one lead 20 may be operably coupled to one electrical signal generator 10 or one extension 30 or that more than one extension 30 may be operably coupled to one electrical signal generator 10. It will be further understood that lead 20 may be coupled to electrical signal generator 10 without use of extension 30 or adaptor.

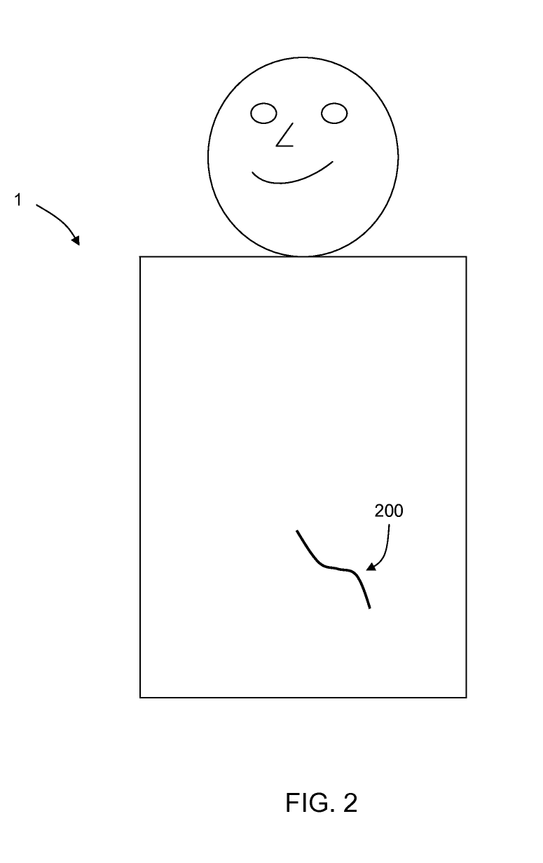

Referring to FIG. 2, a generic painful region or scar 200 of a patient 1 is shown. The painful region or scar 200 may be any scar, such as a surgical scar. For example, the scar may be a scar associated with a herniorrhaphy, a surgical procedure used to correct hernia, a scar associated with a thoracotomy, back surgery, hysterectomy, or a caesarean incision, or the like. While the scar 200 is depicted as being on the torso of the patient 1, it will be understood that the scar may be located at any location of the patient 1. It will be further understood that regions of pain other than scars may be effectively treated by employing the methods, devices and systems described herein. However for the purposes of brevity, the remainder of this disclosure will focus primarily on scars.

Electrodes of one or more lead may be implanted to bracket a scar in any suitable manner.

In various embodiments, electrodes are implanted subcutaneously in the patient along an arcuate path. For example, the arcuate path may be wishbone-shaped, circular or semi-circular, elliptical or semi-elliptical, “U” shaped, or the like. By bracketing the region of pain; e.g. a painful scar, in such a manner, more effective paresthesia may be produced to provide pain relief when therapeutic electrical signals are applied via the electrodes.

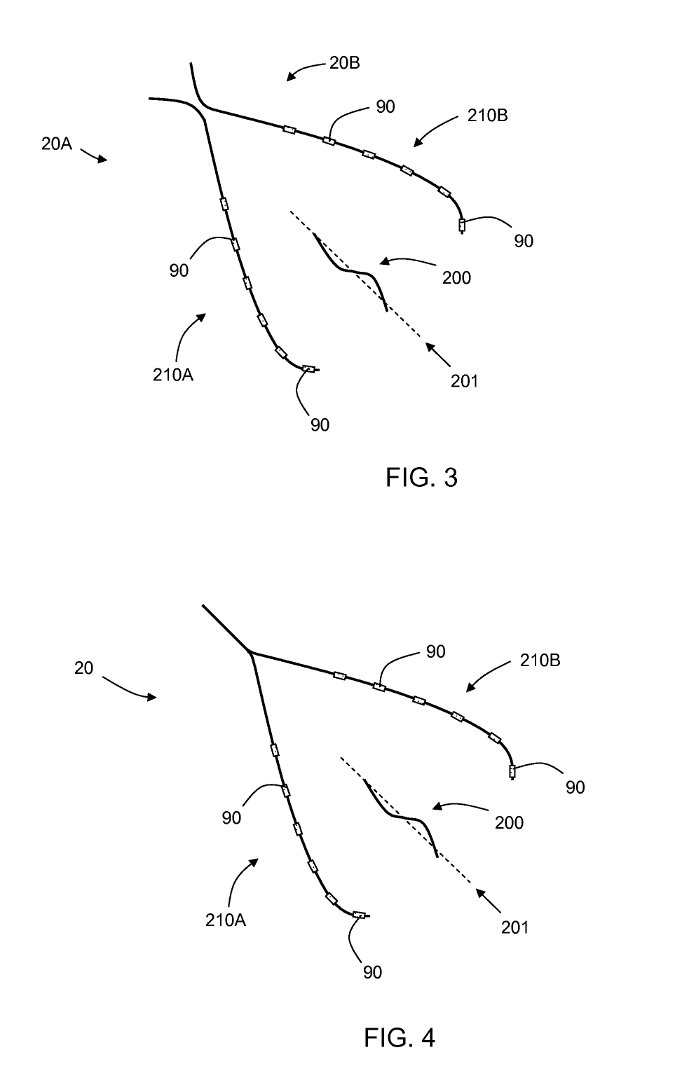

Referring now to FIGS. 3-5, some example of electrode 90 implant configurations that may be employed for effectively bracketing a region of pain 200, such as a scar, are shown. In FIGS. 3-5, electrodes 90 of one or more leads are implanted along an arcuate path to bracket a scar 200. More specifically, the electrodes 90 are positioned such that at least one electrode 90 is positioned on one side of a longitudinal axis 201 of the scar 200 and at least one electrode is positioned on a generally opposing side of the longitudinal axis 201 of the scar 200. Preferably a plurality of electrodes 90 are positioned on each side of the longitudinal axis 201 of the scar 200. The longitudinal axis 201 of a scar 200 may be considered to be a line running through the center line (or best fit center line) of the scar. The longitudinal axis can be determined at the surface of the patient's skin.

By implanting the electrodes 90 along an arcuate path to bracket the scar 200, the distance between electrodes 90 on either side of the scar 200 or longitudinal axis 201 may vary so that electrodes at optimal distances from the scar 200 may be used for application of electrical therapy to treat the pain. Further, the arcuate path provides electrode 90 spread across a wider area than a linear path, which may result in the ability to capture nerves that might not be captured with stimulation using straight line implanted electrodes. Thus, electrical signals may be applied to a large area around the scar 200 through one or more of the plurality of electrodes 90 to produce parasthesia for pain relief. Often, pain in the region of the scar is due, at least in part, to mechanical injury (cut or transaction) of all or part of a nerve. A wishbone or other shape that brackets or encompasses the scar might have the advantage of targeting an injured nerve trunk as it innervates the region that was damaged due to the surgical incision.

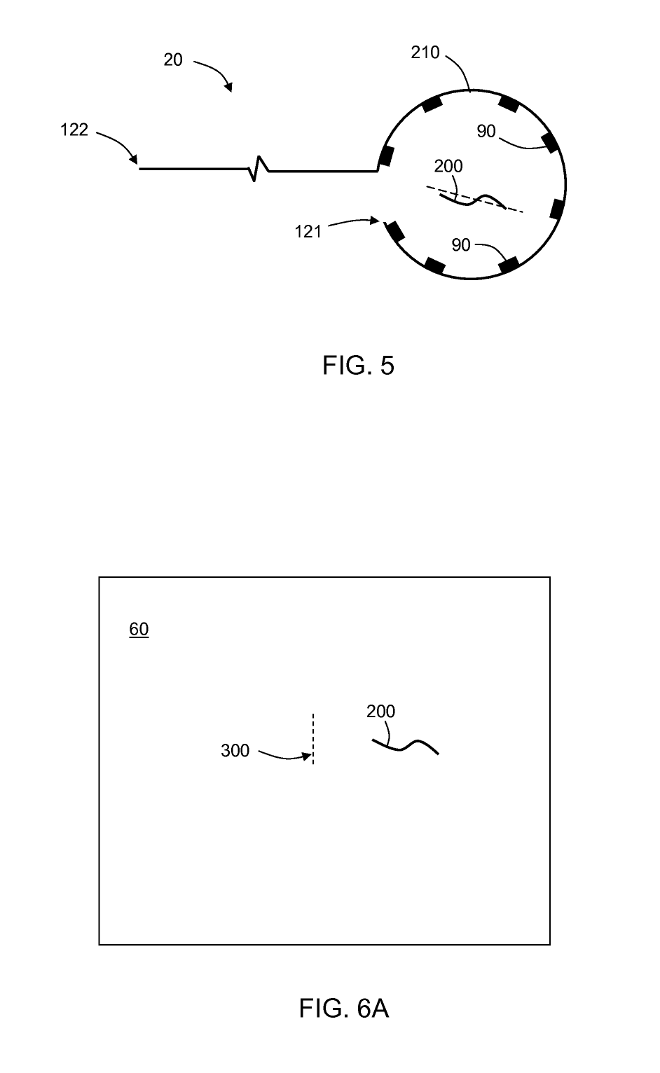

Referring specifically, to FIGS. 3-5, one or more leads 20 (20A, 20B) may be employed to bracket a region of a scar 200. For example, FIG. 3 shows two leads 20A, 20B implanted along arcuate paths so that electrodes 90 at the distal portions 210A, 210B of the leads 20A, 20B bracket the scar 200. FIG. 4 shows a bifurcated lead 20 having two distal ends 210A, 210B implanted along arcuate paths so that electrodes 90 bracket the scar 200. FIG. 5 shows a single lead 20 having a distal portion 210 implanted along an arcuate path so that electrodes 90 bracket the scar region 200. In FIGS. 3-4 the electrodes 90 are implanted in a wishbone-shaped manner. In FIG. 5, the electrodes 90 are implanted in a semi-circular manner. However, it will be understood that the electrodes 90 may be implanted in any suitable arcuate manner to bracket a scar region 200.

Leads may be implanted in the configurations described and contemplated herein through any suitable procedure using any suitable apparatuses. For example, the leads may be implanted percutaneously using a steerable introducer, a curved needle or an arcuate introducer (e.g., as described in PCT Patent Application, filed on even date herewith, entitled ARCUATE INTRODUCER, and having Attorney Docket No. P0035903.01 and claiming priority to U.S. Provisional Patent Application No. 61/218,697, filed Jun. 19, 2009, which applications are hereby incorporated herein by reference in their respective entireties to the extent that they do not conflict with the present disclosure), or the like.

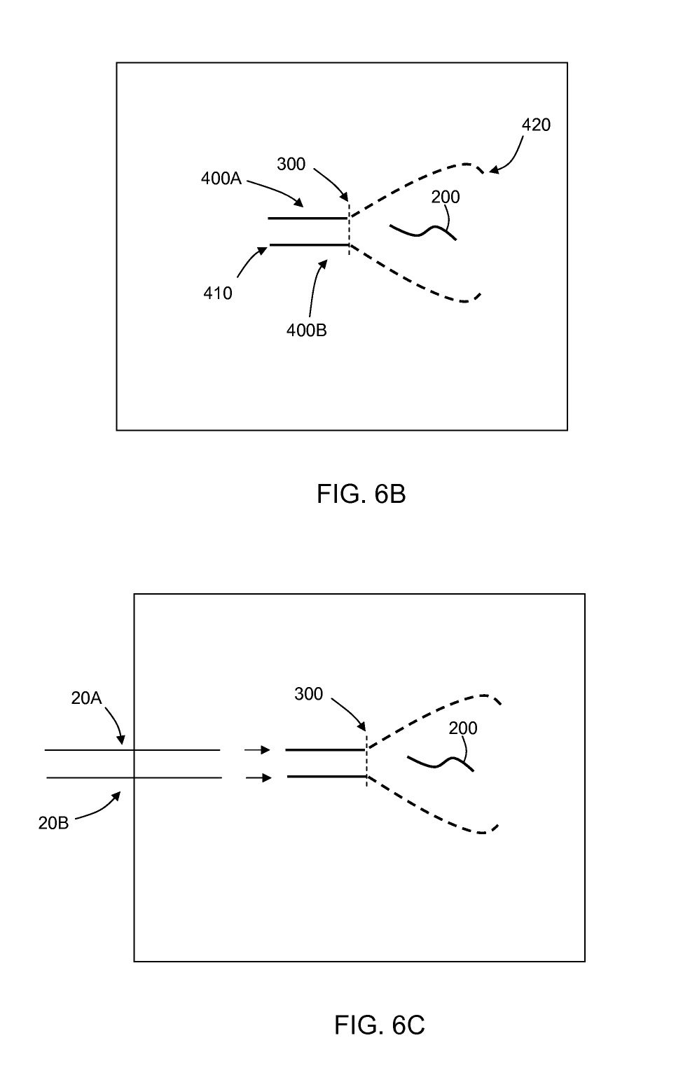

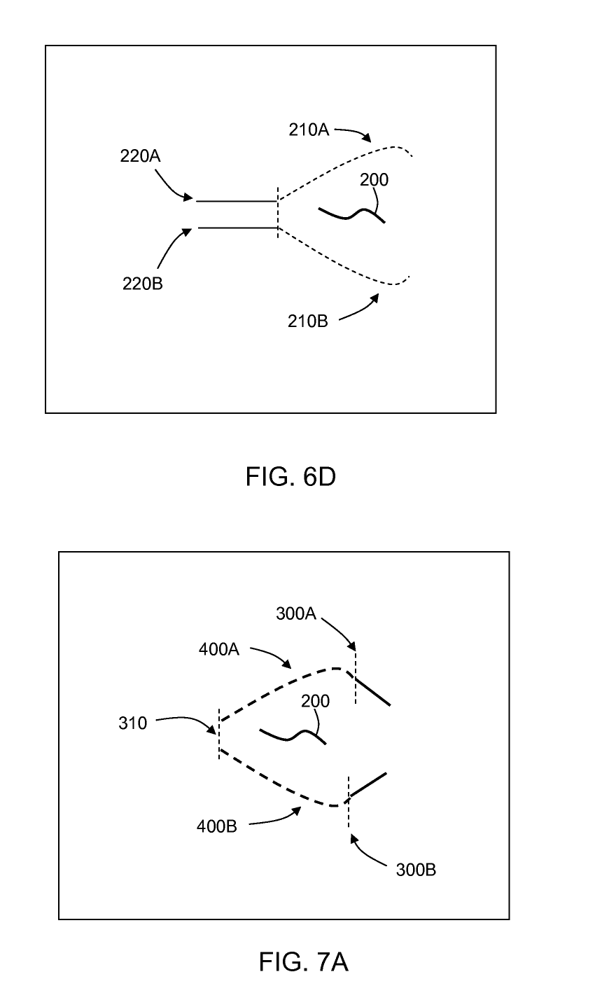

Some examples of methods for implanting leads in an arcuate manner to bracket a region of a scar are shown in FIGS. 6-8. As shown in FIGS. 6A-D, an incision 300 through the skin 60 of a patient may be made in proximity to a painful region or scar 200 (FIG. 6A). Introducers 400A, 400B can be inserted into the incision and tunneled subcutaneously to bracket the scar 200. Of course, in some embodiments, the introducers 400A, 400B may be percutaneously introduced into patient without making an incision. In some embodiments, a guidewire (not shown) is used to tunnel an appropriate arcuate path under the skin 60 of the patient. An introducer 400A, 400B may be fed over the guidewire so that it follows the appropriate subcutaneous path. The guidewire may be removed for insertion of the lead or the introducer may have more than one lumen, and the lead may be introduced through a second lumen. Alternatively, the introducer 400A, 400B may be a steerable catheter, as known in the art, having a lumen through which a lead may be inserted. Alternatively, the introducer may be a curved needle or arcuate introducer that may be pushed through the subcutaneous tissue to create a desired arcuate subcutaneous path. Regardless of the form of the introducer, any suitable method may be used to track the path of the introducer 400A, 400B under the patient's skin, including palpation of the lead beneath the surface of the skin, visualization of the lead underneath the skin, or other visualization techniques such as ultrasound. Once in place, a proximal end of the introducer 400A, 400B is external to the patient and a distal portion is implanted subcutaneously along an arcuate path.

Referring now to FIG. 6C, a lead 20A, 20B may be inserted into a lumen of an implanted introducer 400A, 400B and advanced, typically until the distal end of the lead 20A, 20B generally reaches the distal end of the introducer 400A, 400B. The introducer 400A, 400B may then be withdrawn from the patient over the lead 20A, 20B, leaving the lead implanted along an arcuate path in the patient (FIG. 6D), with the distal end portion 210A, 201B of the leads implanted and the proximal ends 220A, 220B exiting the patient. The proximal ends 220A, 220B may be implanted via standard techniques and tunneled to a location of an implanted electrical signal generator. Alternatively, the proximal ends may be operably coupled to an external electrical signal generator.

In the embodiment depicted in FIGS. 6A-D, two introducers 400A, 400B are shown implanted prior to introduction of leads 20A, 20B. Of course, one introducer may be used to introduce both leads or one lead may be introduced before the second introducer is implanted and the second lead is introduced.

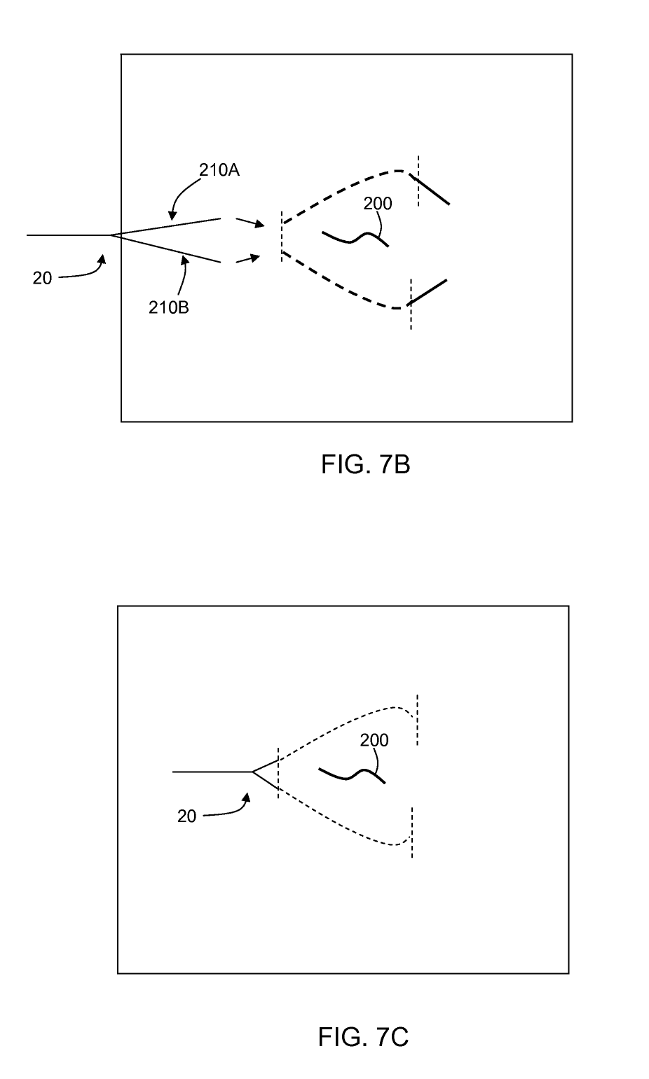

Referring now to FIGS. 7A-C, a method for implanting a bifurcated lead 20 along an arcuate path is shown. First 300A and second 300B incisions are made through the patient's skin 60, and introducers 400A, 400B are tunneled subcutaneously along an arcuate path to bracket a scar 200. Of course, in some embodiments, the introducers 400A, 400B are introduced percutaneously and tunneled subcutaneously without making the first 300A and second 300B incisions. A third incision 310, and possibly a fourth incision (depending on whether the distal ends of the introducers are in proximity to each other or apart from each other), is made so that distal ends 201A, 210B of the lead 20 may be attached to the distal ends of the introducers 400A, 400B, which are implanted subcutaneously (see FIG. 7B). The distal ends 210A, 210B may be pulled through the arcuate subcutaneous paths created by the introducers 400A, 400B when the introducers are withdrawn from the patient (see FIG. 7C). Of course, both distal ends 210A, 210B of the bifurcated lead 20 may be introduced with one introducer or one distal end 210A of the bifurcated lead 20 may be introduced first and the other distal end 210B second, rather than simultaneously using two different introducers 400A, 400B as depicted. It will be understood that introducers having lumens may be used and the distal ends of the leads may be fed proximally through the lumen of the introducer to achieve a similar effect to pulling the lead through the subcutaneous tissue.

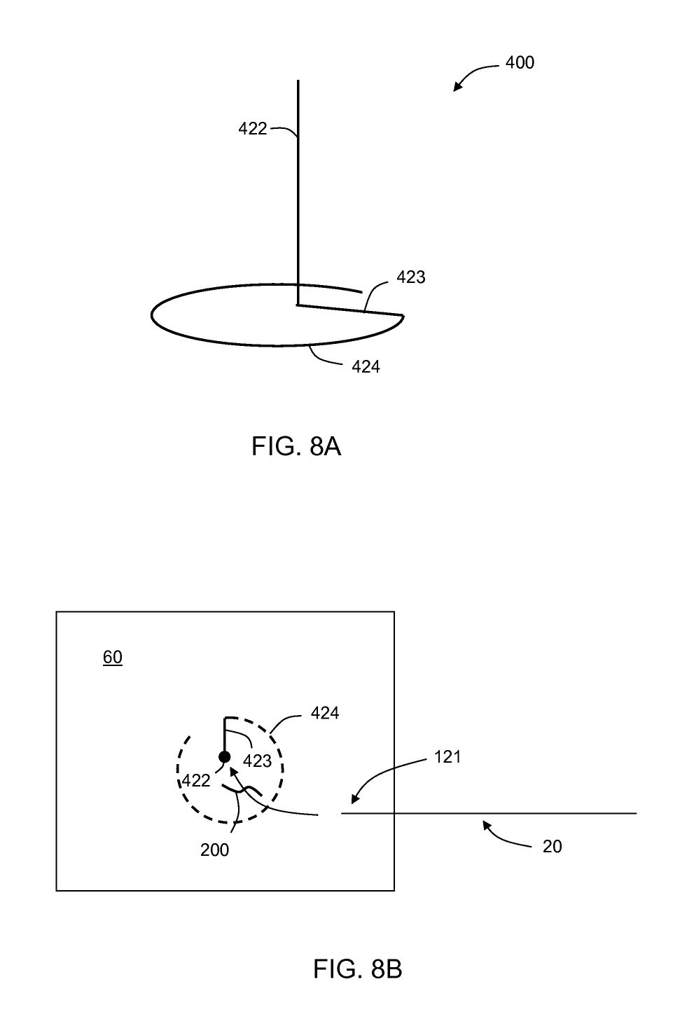

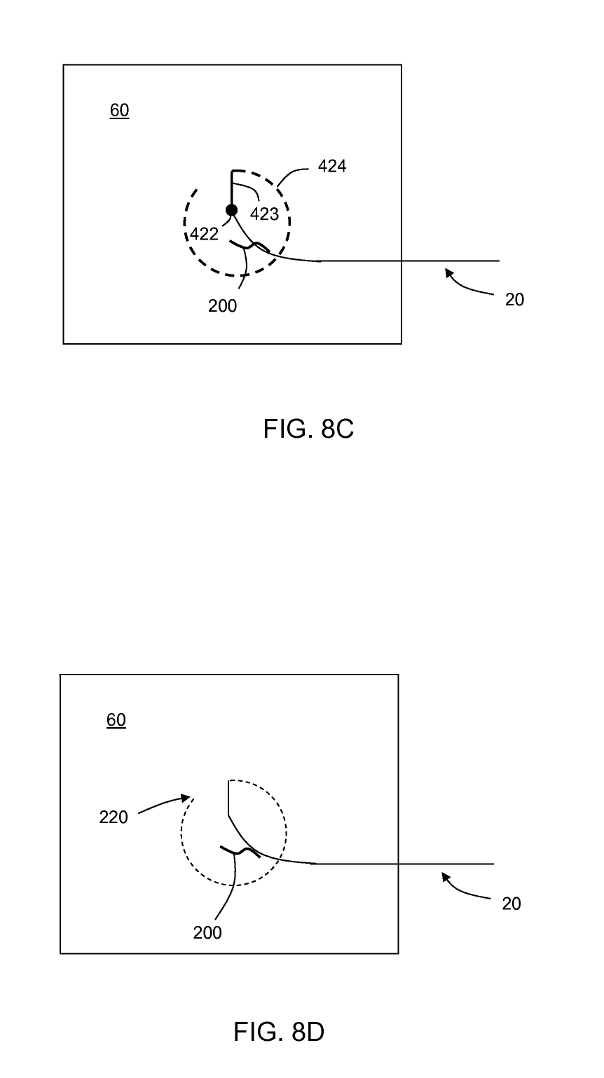

Referring now to FIGS. 8A-D, an arcuate introducer as taught by PCT Patent Application, filed on even date herewith, entitled ARCUATE INTRODUCER, and having Attorney Docket No. P0035903.01 and claiming priority to US Provisional Patent Application No. 61/218,697, filed Jun. 19, 2009, is shown, as well as a method for introducing a lead to bracket a scar. Briefly referring to FIG. 8A, an introducer 400 includes a radial component 423 connecting a shaft 422 to an arcuate component 424. A lumen (not shown) for receiving a lead extends through at least a portion of the shaft 422, the length of the radial component 423 and the arcuate component 424. For additional details regarding such an introducer, see the co-pending PCT application.

Referring now to FIG. 8B, the introducer 400 shown in FIG. 8A is shown percutaneously implanted in a patient, with dashed lines indicating portions under the patient's skin 60. As shown in FIG. 8B-C, a lead 20 may be inserted into the lumen of the introducer. The introducer may then be withdrawn from the patient, leaving the lead 20 implanted along the arcuate path created by the introducer (FIG. 8D).

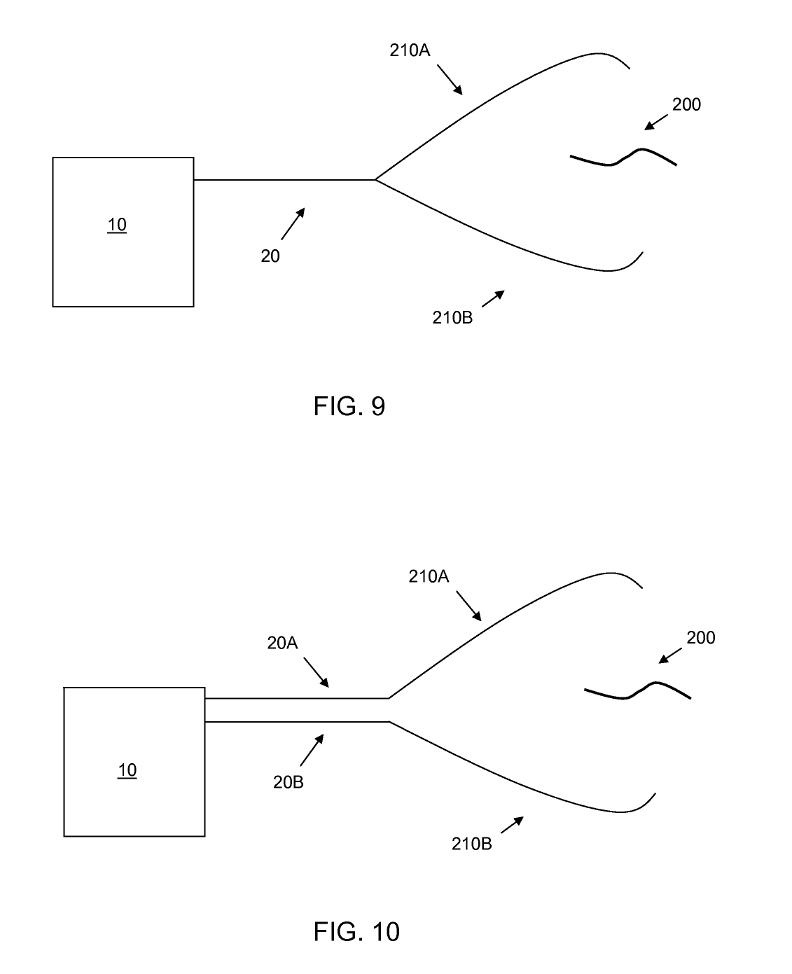

Referring now to FIGS. 9-12, various embodiments of systems that may be used to treat pain associated with a scar are shown. In FIGS. 9-12, the systems include an implantable electrical signal generator 10. However, it will be understood that an external signal generator may be employed in association with the teachings presented herein. In FIGS. 9-12, distal portions 210A, 210B of a lead or leads are implanted subcutaneously in a manner configured to bracket a scar 200, such as described above with regard to FIG. 3-8.

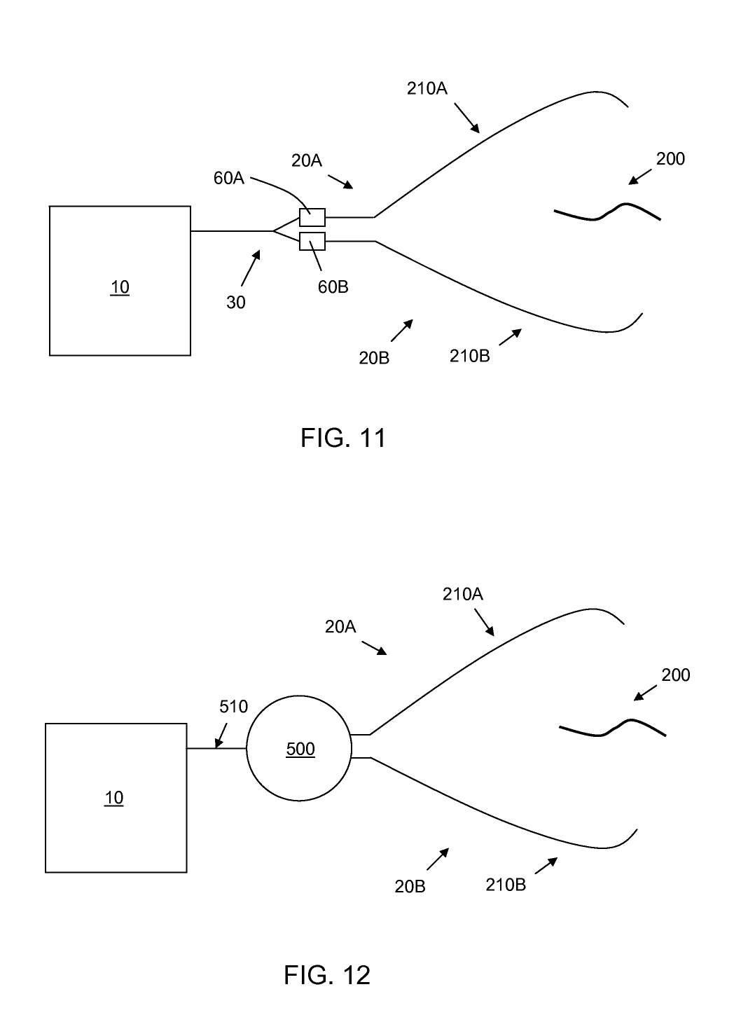

In FIG. 9 a bifurcating lead 20 is directly coupled to the signal generator 10. In FIG. 10, two leads 20A, 20B are directly coupled to the signal generator 10. In FIG. 11 a bifurcating lead extension 30 having two connector regions 60A, 60B for operably coupling independently to two leads 20A, 20B is shown. The lead extension 30 is connected to the signal generator 10. In FIG. 12, two leads 20A, 20B are coupled to a hub 500 configured to operably couple two or more leads to the signal generator 10. The hub 500 is coupled to the signal generator 10 via cable 510, lead, wire, or the like. Any suitable hub 500 may be employed. In some embodiments, the hub 500 is a hub as described in PCT Patent Application, filed on even date herewith, entitled HUB FOR IMPLANTABLE MEDICAL LEADS, and having Attorney Docket No. P0035878.01, and claiming priority to U.S. Provisional Application No. 61/218,452, filed on Jun. 19, 2009, and having Attorney Docket No. P0035878.00, which applications are incorporated herein by reference in their entireies to the extent that they do not conflict with the present disclosure. If it is desirable to use more than two leads to bracket an area of a scar, a hub may be advantageously employed.

It will be understood that electrical signal parameters may be varied as desired for treating pain. Typically, the frequency, amplitude or pulse width of an electrical signal may be varied. An electrical signal having any suitable frequency for treating pain may be used to treat pain as described herein. For example, an electrical signal may have a frequency of about 0.5 Hz to 500 Hz (e.g., about 5 Hz to 250 Hz or about 10 Hz to 50 Hz). For example, the amplitude may be about 0.1 volts to 50 volts (e.g., about 0.5 volts to 20 volts or about 1 volt to 10 volts); for devices that the amps rather than voltage, one skilled in electronics understands the conversion from volts to amps for stimulation devices. An electrical signal may have any suitable pulse width. For example, the signal may have a pulse width of 10 microseconds to 5000 microseconds (e.g., about 100 microseconds to 1000 microseconds or about 180 microseconds to 450 microseconds). For some patients with some devices, the determination of the optimal location and parameters for stimulation occurs within days, for others, within hours or minutes.

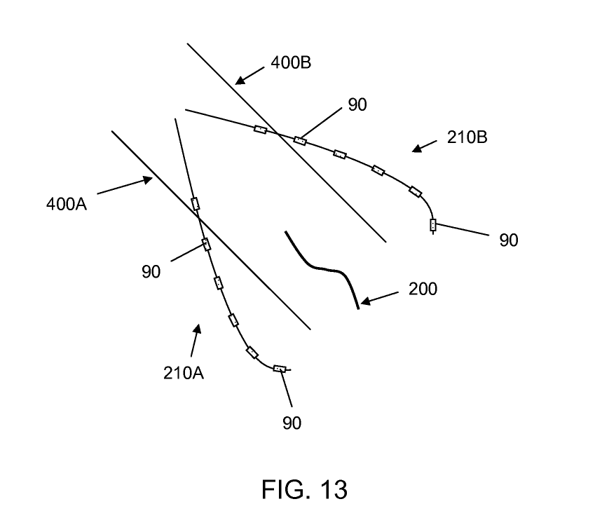

Referring now to FIG. 13, distal portions 210A, 210B of a lead or leads that are implanted subcutaneously are shown bracketing a scar 200. One or more of the electrodes 90 of the distal portions 210A, 210B are in electrical communication with (e.g., able to apply an electrical signal to) one or more nerve trunks 400A, 400B. In situations where a nerve trunk 400A, 400B carries pain signals associated with the scar pain, it may be desirable to capture the nerve trunk within an electrical field generated by one or more of the electrodes to facilitate pain inhibition. Often the ilioinguinal or iliohypogastric nerves are superficial in regions in proximity to scars associated with herniorrhaphies. Accordingly, it may be desirable to position the lead or leads such that electrical signals may be applied to these nerves when treating pain associated with herniorrhaphies.

In the embodiments shown in FIG. 13, the leads or distal portions thereof are shown as being implanted in a generally wishbone-shaped configuration to bracket the scar and positioned to capture the nerve trunks. However, it will be understood that the leads may be implanted such that the electrodes form any other suitable configuration, such as those described above, including semi-circular, oval, “U-shaped or the like.

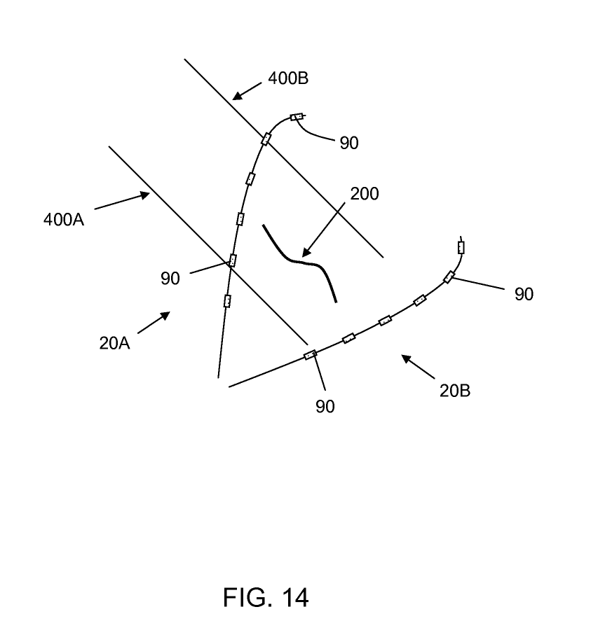

Referring now to FIG. 14, an embodiment where leads 20A, 20B, or distal portions thereof, are implanted transverse to (generally perpendicular to in the depicted embodiment) a longitudinal axis of region of pain or scar 200. Electrodes 90 of the leads 20A, 20B are positioned such that some electrodes 90 are on one side of the axis of the scar 200 and some electrodes are on the opposing side of the axis of the scar 200.

Depending on the size of the region of pain or scar, it may be possible to obtain better electrode coverage with regard to a nerve trunk when leads are positioned transverse or perpendicular to the region of pain or scar. Of course, one or more lead may be implanted in any suitable manner such that at least one electrode is on one side of a region of pain or scar and at least one electrode is on an opposing side or the region of pain or scar.

Thus, embodiments of BRACKETING SCAR FOR TREATING PAIN WITH ELECTRICAL STIMULATION are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A method comprising:
implanting one or more leads in proximity to a scar of a patient, wherein the one or more leads have a plurality of electrodes, wherein the one or more leads are implanted such that a first electrode of the plurality of electrodes is implanted subcutaneously on a side of a longitudinal axis of the scar and a second electrode of the plurality of electrodes is implanted on an opposing side of the longitudinal axis of the scar; and
applying electrical signals to tissue in the region of the scar via the first and second electrodes.

2. The method of claim 1, wherein the plurality of electrodes are implanted along one or more arcuate path.

3. The method of claim 2, wherein one lead comprises the plurality of electrodes.

4. The method of claim 2, wherein the lead is implanted by insertion through a lumen of an arcuate introducer having a shaft, a radial component and an arcuate component, wherein the shaft is coupled to the arcuate component by the radial component, and wherein the lumen extends through the arcuate component, the radial component, and at least a portion of the shaft.

5. The method of claim 2, further comprising pulling the lead through tissue of the patient by a distal end of an arcuate introducer having a shaft, a radial component and an arcuate component, wherein the shaft is coupled to the arcuate component by the radial component.

6. The method of claim 5, further comprising:
operably coupling a distal end of the lead to the distal end of the arcuate component of the introducer; and
withdrawing the introducer from the patient to pull the lead along the arcuate path.

7. The method of claim 1, wherein the scar is a surgical scar.

8. The method of claim 1, wherein the scar is a scar resulting from a herniorrhaphy.

9. The method of claim 1, wherein two leads comprise the plurality of electrodes, and wherein the leads are implanted so that the electrodes form a wishbone configuration bracketing the at least a portion of the scar.

10. The method of claim 1, wherein the electrodes are implanted in a U-shaped configuration bracketing the at least a portion of the scar.

11. The method of claim 11, wherein one lead comprises the electrodes.

12. The method of claim 1, wherein the first electrode is on a first lead and the second electrode is on a second lead.

13. The method of claim 1, wherein the first electrode is on a first distal arm of a bifurcating lead and the second electrode is on a second distal arm of the bifurcating lead.

14. A method for treating pain associated with a scar, comprising:
implanting one or more leads in proximity to the scar of a patient, wherein the one or more leads have a plurality of electrodes, wherein the one or more leads are implanted such that a first electrode of the plurality of electrodes is implanted subcutaneously on a side of a longitudinal axis of the scar and a second electrode of the plurality of electrodes is implanted on an opposing side of the longitudinal axis of the scar; and
applying electrical signals to tissue in the region of the scar via the first and second electrodes to treat the pain.

15. A method for treating post-herniorraphy pain, comprising:
implanting one or more leads in proximity to a post-surigical herniorappathy scar of a patient, wherein the one or more leads have a plurality of electrodes, wherein the one or more leads are implanted such that a first electrode of the plurality of electrodes is implanted subcutaneously on a side of a longitudinal axis of the scar and a second electrode of the plurality of electrodes is implanted on an opposing side of the longitudinal axis of the scar; and
applying electrical signals to tissue in the region of the scar via the first and second electrodes to treat the pain.

16. A method according to claim 15, wherein the one or more leads are implanted such that at least one of the plurality of electrodes is in electrical communication with a trunk of the ilioinguinal nerve or the ilohypogastric nerve.
